(19) United States
(12) Patent Application Publication
(10) Pub. No.: US 2008/0033369 A1
(43) Pub. Date: Feb. 7, 2008

(54) REAL-TIME DISPLAY FOR A DEVICE FOR THE DOSED ADMINISTRATION OF A PRODUCT

(76) Inventors: Philippe Kohlbrenner, Kaltacker (CH); Peter Stettler, Kirchberg (CH); Christoph Meier, Utzenstorf (CH); Martin Wittwer, Bowil (CH); Thomas Schuler, Sumiswald (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/769,227

(22) Filed: Jun. 27, 2007

Related U.S. Application Data
(63) Continuation of application No. PCT/CH2005/000711, filed on Nov. 30, 2005.

(30) Foreign Application Priority Data
Dec. 31, 2004 (DE) 102004063664.8

(51) Int. Cl. A61M 5/178

(52) U.S. Cl. 604/207

(57) ABSTRACT

An injection device including a regulating element, a sensor, an electronic evaluation system connected to the sensor, and a display connected to the evaluation system for displaying information, wherein the sensor is arranged in the injection device or coupled to operational elements of the device whereby, during a delivery or operational process, the dose still to be delivered can be assessed. A method for displaying a dose of a substance to be delivered is encompassed by the present invention, the dose being detected and displayed during a regulating or operating process associated with an injection device, wherein an amount or dose of the substance still to be delivered is displayed during a delivery process.

### CROSS-REFERENCE TO RELATED APPLICATIONS

This application is a continuation of International Patent Application No. PCT/CH2005/000711, filed on Nov. 30, 2005, which claims priority to German Application No. DE 10 2004 063 664.8, filed on Dec. 31, 2004, the contents of both of which are incorporated in their entirety herein by reference.

### BACKGROUND

The present invention relates to device for dispensing, delivering, injecting, infusing or administering substances, and to methods of making and using such devices. More particularly, it relates to a device for administering doses of a product, such as an injection device or pen for example, by which a metered quantity of a liquid product such as insulin, growth hormone or osteoporosis preparations, for example, can be self-administered by a user.

The functional elements of an electronic or mechanical real-time display and an electronic life timer described below may be used in conjunction with a suitable injection device, e.g., an injection pen of the type described in the patent application entitled “Device for administering doses of a liquid product” owned by the owner of the present application. An injection pen in accordance with the present invention may differ on the basis of the dose display and/or as regards the sensors, electronic systems or display elements which will be described below.

Due to the strain and mechanical stress which occurs when using an injection pen and due to ageing processes, it may be that the components of the injection pen, such as springs or components which are moved often for example, are altered or impaired, thereby impairing correct operation and the ability of the pen to function.

When a user sets a dose or quantity of a substance to be dispensed by the injection device, for example by forcing the substance out of an ampoule inserted in the injection device, it is of advantage to the user if he can take a reading of the currently set dose on a display of the injection device. During and after setting the dose in known injection devices, the set dose can be displayed and a user can initiate the injection process by depressing a trigger button for example and, after the injection process is complete the displayed dose is turned or runs back from the set dose to zero or is re-set.

### SUMMARY

One object of the present invention is to provide a device and a method by which the service life of an injection device, e.g., an injection pen, can be determined and the end of the service life indicated by a display.

Another object of the present invention is to provide a device and a method, whereby a user of an injection device is provided with information about and/or for implementing an injection process that has been interrupted or otherwise affected.

In one embodiment, the present invention comprises an injection device comprising a regulating element, a sensor, an electronic evaluation system connected to the sensor, and a display connected to the evaluation system for displaying information, wherein the sensor is arranged in the injection device or coupled to operational elements of the device whereby, during a delivery or operational process, the dose still to be delivered can be assessed. A method for displaying a dose of a substance to be delivered is encompassed by the present invention, the dose being detected and displayed during a regulating or operating process associated with an injection device, wherein an amount or dose of the substance still to be delivered is displayed during a delivery process.

An injection device in accordance with the present invention has at least one sensor for detecting at least a setting or a deployment of the injection device. For example, the sensor may be provided in the form of a mechanical switch or a pressure switch on the injection device, for example in the region of a dose setting element or an injection knob, to detect when the injection device is being used for the first time or when an ampoule is inserted. It is possible to provide a sensor or switch element at another point of the injection device, such as in the region of a reservoir in which an ampoule is inserted, or coupled via a connecting element with a component of the injection device, e.g., to detect when an ampoule is inserted. A sensor may also be provided in the form of an encoder or rotary sensor, which can be coupled with a dose metering element or a plunger or threaded rod of the injection device, for example, by means of a gear meshing action via a spur gear, a ring gear or a gear mounted approximately parallel with the mid-axis or injection device, for example, to detect a setting or dispensing operation of the injection device. In principle, any sensor may be used for the purpose of the invention which is suitable for detecting the first time the injection device is operated or deployed, or by which a use or operation of the injection device can be detected, for example to set a dose to be dispensed, for assembling the injection device, for preparing for an injection, implementing an injection, dispensing a set dose or for changing an ampoule. Using one or more such sensors for qualitatively or also quantitatively detecting operating procedures, operating or status parameters, it is possible to detect when an injection device is being used or deployed for the first time to start a timer or timer clock provided in the injection device which emits a signal after a selected, fixed period, e.g., three years, which can be set by the manufacturer to indicate that the service life of the injection device for which the manufacturer of the injection device offers a guarantee of correct operation of the injection device has expired.

In some embodiments, such a timer or a time element may be based on mechanical or chemical principles in which a chemical substance may be provided which changes colour after a pre-definable time so that a user can tell from the colour of the substance whether the injection device has already exceeded the specified service life or not.

In some preferred embodiments, such a timer or a time control system may be operated by an electronic circuit, which is connected to at least one of the sensors described above and receives a start signal from one or more of these sensors the first time the injection device is used or when a full ampoule is inserted to start the timer. On expiry of a period, which might be set by the manufacturer or predefined or set by a user or calculated by the circuit taking account of the state of stress of the injection device, a signal is emitted by the circuit to a display or a lamp to indicate to a user that the injection device has reached the end of its service life.

When the timer has run its sequence and it has been ascertained that the service life of the injection device has expired, an optical and/or acoustic and/or perceptible or tactile signal merely has to be generated to indicate to the user that the injection device should no longer be used for safety reasons. In principle, however, the injection device can still be used but an appropriate warning signal can be emitted every time it is used, for example, in the form of red LED or OLED which is illuminated or flashes. Another alternative, after it has been established that the injection device has reached the end of its service life, is for a stop signal to be emitted by an electric or mechanical circuit to an actuator such as an electric motor, a magnetic switch or a catch element, so that the actuator places the injection device in a mode in which it can no longer be used and a dose can no longer be dispensed, in which case a rotatable setting element is blocked, functional components of the injection device which have to be coupled with one another during a setting or dispensing operation are permanently uncoupled or the injection button is prevented from being operated by generating a permanently fixed connection between an injection button and the injection device.

In some embodiments, a data memory with a timer or a clock may also be provided in the injection device in which data relating to the settings or operations of or performed on the injection device can be stored, such as data relating to the time it was first used, the number times of dose setting, dispensing operations, and/or the number of times the ampoule was changed. A processor and/or controller provided in the injection device can compare the data relating to the use of the injection device stored in it with threshold values permanently set or pre-fixed in the injection device by the manufacturer for the maximum permissible use of the injection device so that when the maximum permissible number of ampoule changes or dispensing operations has been reached or exceeded, a signal can be emitted to generate a warning signal as mentioned above, thereby indicating to the user that that maximum number of ampoule changes or dispensing operations has been reached or exceeded. If the control or operating parameters of the injection device are used for assessing and/or determining the end of the service life of the injection device, the period of the service life may differ depending on how the injection device has been used and deployed by a user.

It is also possible for the end of the service life to be determined taking account of different parameters, in which case the service life of an injection pen that has not been used terminates after a pre-set period, e.g., three years, and each operation of the injection device detected by one or more sensors, such as an ampoule change or dispensing operation is deducted from a pre-set time for the maximum service life and a pen that has been used frequently since it was first used emits a signal indicating that the end of the service life has been reached earlier than a pen that has not been used frequently.

If the service life display or the life timer is controlled on the basis of a number of ampoule changes detected by a sensor, a so-called system check may additionally be run. If the ampoule is full, a switch of the life timer is operated by the threaded rod and a connecting piece, as a result of which the LED is briefly illuminated with green light.

In some preferred embodiments, the indication to a user that the service life has come to an end may be effected by an optical display and may be provided by or in conjunction with the electronic or mechanical display used to display a set dose. The optical display may be provide by an LCD display, and in addition or as an alternative, two or more counting rings or counting dials may be provided, at which a signal is directly generated or set, indicating to a user that the service life has reached an end. A lamp, such as an LED or OLED may be provided which is illuminated or flashes for the first time or is illuminated with a differently coloured light when the injection device has reached the end of its service life.

A multi-coloured OLED or LED or a red LED may be provided on or in the injection device, which is disposed next to a green LED for illuminating a setting display, so that a user who is accustomed to looking at the setting display can be shown a red light to indicate that the injection device should not be used again. One or more OLEDs or LEDs may be disposed behind an LCD display or inside coaxially disposed and at least partially or totally transparent counting rings or counting dials for the dose display so that the red light of the LED or OLED is able to shine through the transparent counting rings or counting dials. This being the case, another lamp may be provided in the vicinity of or next to a lamp for displaying the service life end, which may be illuminated in a different colour and/or also emits light with a brightness that is different from that of the service life display lamp. For example, a green LED may be illuminated behind an electronic or mechanical display if a sensor has ascertained that a user is operating a setting or operating element of the injection device, as a result of which the user can read the set dose even in darkness or poor light conditions. Once the end of the service life has been detected, this lamp may be switched off permanently or switched so that it emits light of a different colour, so that the service life end display or lamp flashes or is illuminated permanently. Thus, a user would not see the green light he usually sees during a setting operation, but can tell from the red light generated and/or from a flashing light that the injection device has reached the end of its service life and should therefore no longer be used, even though it is possible to continue using the injection device in principle.

A battery may be provided as a power supply to the sensors, circuits, controllers, processors, memory or displays associated with the injection device. The battery may be any suitable battery such as a button battery or an accumulator which can be charged, in which case the optical display elements described above can also be used to indicate to a user battery state or that the accumulator needs to be recharged.

The present invention further relates to a method of determining the service life of an injection device, whereby a start of the service life period is determined by a sensor which detects a control operation by a user and whereby the sensor generates a signal which sets or defines a time from which the service life period starts to run. Once it has been established that the pre-set service life period or a shorter service life period re-calculated on the basis of uses has expired, an optical and/or acoustic signal can be emitted immediately or when a usage operation is next detected and can indicate to a user the end of the service life.

The present invention further relates to a computer program or an algorithm, by which a program of the type described above can be run, for example a service life calculation taking account of deployments, which reduce the service life, such as injections or ampoule changes, for example.

By virtue of another aspect, the present invention relates to an injection device with a real-time display for continuously displaying a dose ready for dispensing set by the injection device and another dose yet to be dispensed. An injection device in accordance with the present invention containing a substance to be dispensed or in which an ampoule has been inserted has a setting element such as a dose setting knob or a dose metering element, by which a dose for dispensing or for an injection can be set or fixed, e.g., by turning, pushing or pulling out. At least one sensor is also provided, which is connected directly or indirectly to the setting element or to a setting or dispensing mechanism of the injection device, or is coupled via gears, to detect the quantity of a dose of a substance available for dispensing from the injection device. An evaluation unit, such as an electric circuit or a processor, which is connected to the at least one sensor, may determine what dose has currently been set or what dose still has to be dispensed from the injection device on the basis of the setting by the signals from the at least one sensor directly, in other words directly from the signals or from the time sequence of the signals, taking account of previous signals of the at least one sensor. For the purpose of the present invention, the evaluation unit also may determine the quantity of the set dose and that still available for discharging or dispensing but not yet dispensed whilst the substance contained in the injection device is being dispensed. A display connected to the evaluation unit can display the dose set by a user, for example as a numerical value, so that the set dose can be read from an LCD or by mechanically coupled counting gears or rings. In some embodiments, a dose or number counted backwards may be shown on the display as a substance is being dispensed.

Accordingly, a mechanical or electronic display of the injection device will also display the quantity of substance already dispensed or the status of the mechanism as a real-time display.

In some embodiments, if a dispensing operation is interrupted a user can tell what quantity of a substance he has already injected or what dose still has to be injected.

In some preferred embodiments, an electronic unit of the injection device is a modular design and the injection device is therefore designed so that it can be ascertained during assembly whether a mechanical or electronic display is provided and can be inserted or snap-fitted, in which case it is also possible to use different housing shells. However, it is not necessary to modify the injection device to integrate either a mechanical or electronic display in the injection device. In some embodiments, it may be preferred to provide an electronic unit as a component unit for directly assembling in the injection device, in which case plastic parts and the steps needed to assemble them can be dispensed with.

With a real-time display of the type provided by the present invention, both the setting or correction of a dose and the dispensing and, if necessary the correction to a set dose, can be displayed during the dispensing operation.

By continuously measuring or calculating the quantity or substance dispensed during a dispensing operation and/or the substance still available for dispensing and amounting at most to the value of the set dose, a check can be made by an evaluation unit or circuit contained in the injection device or by a user taking a reading from the display to ascertain whether the dispensing operation has proceeded correctly and has already terminated or must still be continued. Accordingly, a user is provided, in real time, in other words immediately, with information enabling him to tell in what operating mode the injection device is, in other words whether a part quantity of a set dose has already been dispensed or what quantity of the set dose has already been dispensed or still has to be dispensed. A user is therefore also able to stop the injection in a controlled and defined manner and continue at a later point in time.

In some embodiments, a sensor for detecting the dose still to be administered is coupled or connected to a setting element or a dispensing mechanism of the injection mechanism, such as the commonly known plunger rods or coupling elements, and detects the operating mode of the injection device or information and parameters instantaneously, such as a rotation angle or the number of rotations of a unit provided in the injection device as a way of setting or dispensing a dose, such as a coupling or toothed rack. In some embodiments, the sensor may be a rotary switch or encoder, which does not have a stop. An encoder of this type is manufactured by Alps Electric Co., Ltd. of Tokyo, Japan for example. With an encoder, a rotary movement of a setting element or dose setting element can be directly detected or the rotation of a coupling sleeve or a spur gear or toothed rack engaging with the coupling sleeve can be detected. The signal detected by the encoder may be processed by an electronic unit contained in the injection device and can be forwarded to an LCD enabling the user to take a reading. Such an encoder may also be used as a sensor for a life timer of the type described above for detecting the start of the service life or a setting or dispensing operation.

The display used to indicate the dose which has still been released for dispensing may be an LCD display, which is provided in an electronic component or an E-module of the injection device and can be lit or illuminated by a lamp or LED as described above. It would also be possible to use a mechanical display in the form of one, two or more counting rings such as described in patent specifications EP 0 554 996 B1 or EP 0 897 728 A1, the teaching of which relating to the mechanical design and coupling of units and tens counter rings is incorporated in this application.

In some embodiments, a real-time display of the injection device may be provided with mechanical elements, in the form of a coaxial display of two or more counting rings lying adjacent to one another, which have numbers “0” to “9” printed on them in the circumferential direction, of the type used to count kilometres in automotive applications or counting systems in cassette players. A display of this type comprising counting rings or counting dials disposed coaxially adjacent to one another may be provided on the injection device coaxially in the rear region of the injection device in the vicinity of a setting knob or dose setting element, and coupled with such a setting knob or dose metering element, so that whenever a dose metering element or dose metering gear is moved, the mechanical display is also rotated when the dose is being set, when the dose is being corrected or during the dispensing operation. The mechanical display provided by the present invention may be coupled with a setting and/or dispensing or discharge mechanism so that the quantity of the dose still to be dispensed is always displayed by the mechanical display, including during the dispensing operation, and the mechanical display therefore counts backwards whilst the substance contained in the injection device is being dispensed.

In some embodiments, the mechanical counting system may be provided with a planet drive for rotating the counting gears. This being the case, a drive disc coupled with a setting element, such as a dose metering element, or with an element of the dispensing mechanism such as a toothed rack or plunger rod, or with a coupling or coupling sleeve, drives one, two, three or more planet gears coupled with the drive disc or mounted on the drive disc. These planet gears engage in teeth provided on the external face of a units counting ring and thus drive the units counting ring. On an inner or outer face of the units counting ring in the circumferential direction, teeth are disposed only within a region corresponding more or less to a unit counting step of 36 degrees, so that the units counting ring is able to drive a coupling or transmission gear meshing in the teeth after every full revolution, which drives a tens counting ring provided coaxially with the units counting ring after a full revolution of the units counting ring so that it is rotated further by a predefined value of 36 degrees to turn a number provided on the tens counting ring forwards or backwards to the position of an adjacent number. An annular gear may be used as a support for the units and tens counting rings and has teeth on its internal face in which the planet gears driving the units counting ring mesh. The coupling gear may also be mounted in the annular gear. The annular gear may be mounted in the housing of the injection device or integrated in it so that it can not turn.

By fixing the distance of the teeth, the translation ratio of the drive disc on the units counting ring can be fixed, in which case the units counting ring can effect 2.4 revolutions for one revolution of the drive disc, although it would also be possible to implement other translation ratios by varying the distance of the teeth accordingly or by opting for different diameters of the mutually meshing gears or circumferentially extending rows of teeth.

The counting rings may be made from a non-transparent material or from a transparent material as described above, so that light from one or more lamps or LEDS disposed inside the counting rings can be emitted to the outside, enabling a user to take a reading of a set dose, even if it is dark.

The present invention further relates to a method of displaying a dose to be dispensed by an injection device after the setting operation, whereby a set dose is detected and displayed, and the quantity of the dose still to be dispensed is continuously measured or determined and displayed during the dispensing and administering operation so that a display counting backwards can be provided during a dispensing operation.

In some preferred embodiments, the display is reset to “0” when the ampoule is changed, when the set dose has been dispensed or when it has been ascertained that another dose can not be dispensed without another setting operation. A sensor may be used to sense the abutment of one component on another component of the injection device once the dispensing operation has been completed. Consequently, a display can be automatically reset to zero after every dispensing operation and/or when the ampoule is changed, to ensure that the display or electronic system of the injection device does not lose any counting steps and is ready for a new setting operation starting from a defined initial state. For example, a mechanical display can be automatically turned back to “0” by means of a drive element such as illustrated in FIGS. 1A to 1C, for example a rotating or helical spring, when the pen is screwed on to change the ampoule.

An electronic display of the injection device and/or a lighting system of an electronic or mechanical display, such as an LED, can be switched on whenever an operation or movement of the injection device is detected by a sensor and can be switched off again when a pre-definable period, e.g., 30 seconds, has elapsed, if it is ascertained that the injection device has not been operated within a pre-definable period or if it is ascertained that a dispensing operation has terminated.

### BRIEF DESCRIPTION OF THE DRAWINGS

FIGS. 1A-1C show views in cross-section of an embodiment of an injection device in accordance with the present invention;

FIGS. 2 and 3 are, respectively, a diagram in cross-section and an exploded diagram showing a sensor for an electronic life timer in the dose setting knob;

FIG. 4 shows an embodiment of an electronic real-time display with a spur gear for coupling the sensor with the setting and dispensing mechanism;

FIGS. 5A-5C illustrate an embodiment of an electronic real-time display with an encoder serving as a sensor, disposed offset from a dose metering element;

FIG. 6 illustrates a coaxial real-time display driven by a planet gear;

FIG. 7 shows a mechanical coaxial real-time display with a life timer;

FIG. 8 is a view in cross-section showing a detail of the injection device illustrated in FIG. 1;

FIGS. 9 and 10 show views in section of the detail illustrated in FIG. 8; and

FIG. 11 is a detailed view of the region denoted by X in FIG. 9.

### DETAILED DESCRIPTION

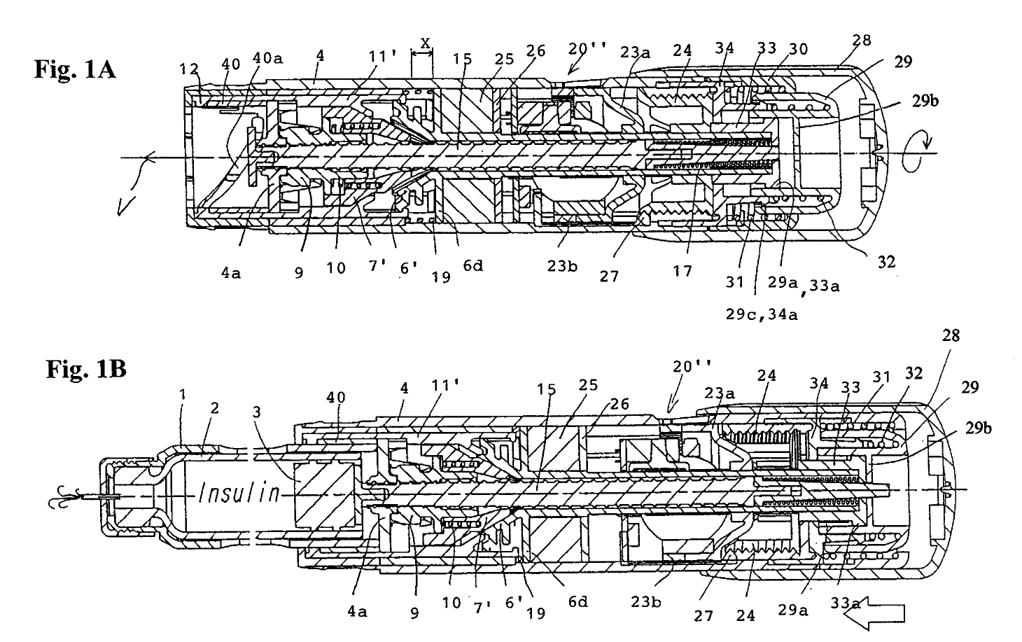

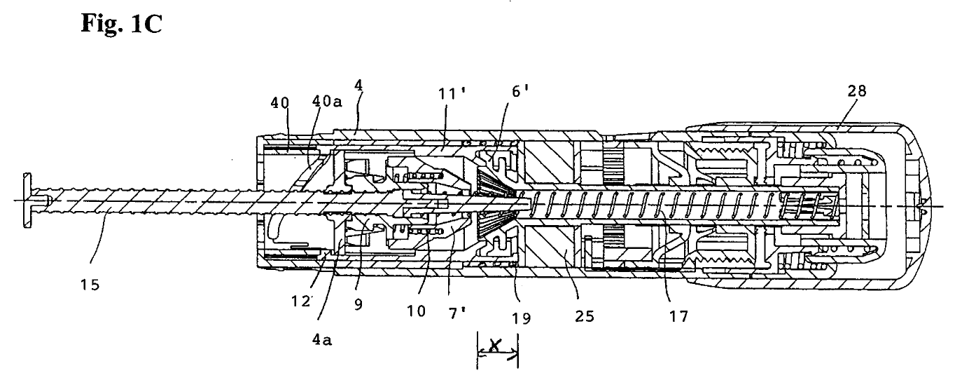

FIGS. 1A-1C illustrate, in cross-section, an embodiment of the present invention comprising an injection device in which a life timer and/or an electronic or mechanical real-time display can be integrated. With regard to fastening, mounting, attaching or connecting the components of the present invention, unless specifically described as otherwise, conventional mechanical fasteners and methods may be used. Other appropriate fastening or attachment methods include adhesives, welding and soldering, the latter particularly with regard to the electrical system of the invention, if any. In embodiments with electrical features or components, suitable electrical components and circuitry, wires, chips, boards, microprocessors, inputs, outputs, displays, control components, etc. may be used. Generally, unless otherwise indicated, the materials for making the present invention and/or its components may be selected from appropriate materials such as metal, metallic alloys, ceramics, plastics, etc.

FIGS. 1A-1C illustrate an embodiment of an injection device in which the driving force for dispensing the product during the administering operation is applied not manually but by a driving element 25, provided in the form of a driving spring. The driving element 25 is a helical spring acting as a rotary spring with spring coils extending about the thread axis of the thread engagement between the coupling output element 9 and the plunger rod 15. The spring coils are disposed radially with respect to the thread axis one above the other; their pitch relative to the thread axis is zero. An inner end of the spring coils is attached to the coupling input element 6′ and another end is connected to a fixing structure 26 which can be moved with the housing part 4 in the direction of the coupling movement X but is prevented from turning. The fixing structure 26, on the other hand, is connected to the coupling input element 6′ so that it can not be moved in and opposite the direction of the coupling movement X. The coupling input element 6′ can be rotated relative to the fixing structure 26 about the thread axis.

The coupling sleeve 8′ may be present or not. If not, the coupling interconnecting element 7′ engages directly with the coupling output element 9 so that the rotary driving movement of the coupling input element 6′ is transmitted to the coupling output element 9.

The retaining mechanism in the embodiment illustrated comprises a resetting element 19, as well as the coupling mount which in turn forms the linear guide 4a for the plunger rod 15, and a support structure 6d which is connected to the coupling input element 6′ so that it can not be moved at least in and opposite the direction of the coupling movement X. In the embodiment illustrated as an example, the coupling input element 6′ and the support structure 6d comprise a single piece. The driving spring 25 is axially surrounded by the support structures 6d and 26. The resetting element 19 is supported on the housing part 4 in the direction of the coupling movement by the coupling mount and on the support structure 6d opposite the direction of the coupling movement X. The resetting element 19 transmits an elastic return force acting against the coupling movement X to the coupling input element 6′. It in turn acts as a compression spring.

The compensating spring 17 biased between the plunger rod 15 and the connecting part 33 supports the resetting element 19. The compensating spring 17 could be used disengage the coupling members 6′, 7′ and 9 and, in principle, replace the resetting element 19. However, in some preferred embodiments, it is so weak that it can no longer retain the coupling members 6′, 7′, 9 in the retained position with sufficient reliability and thus maintain the uncoupled state after it has at least partially relaxed.

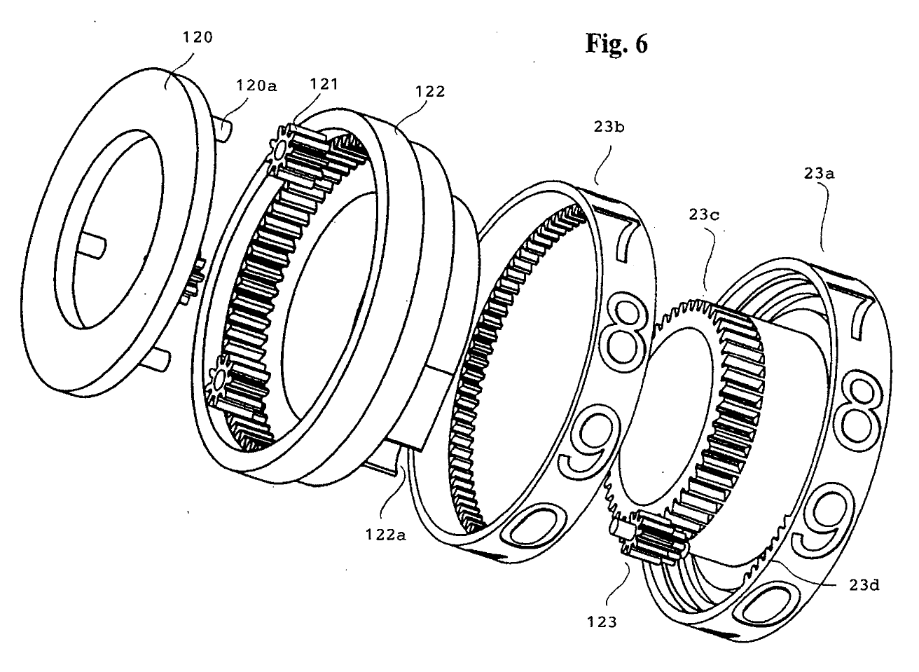

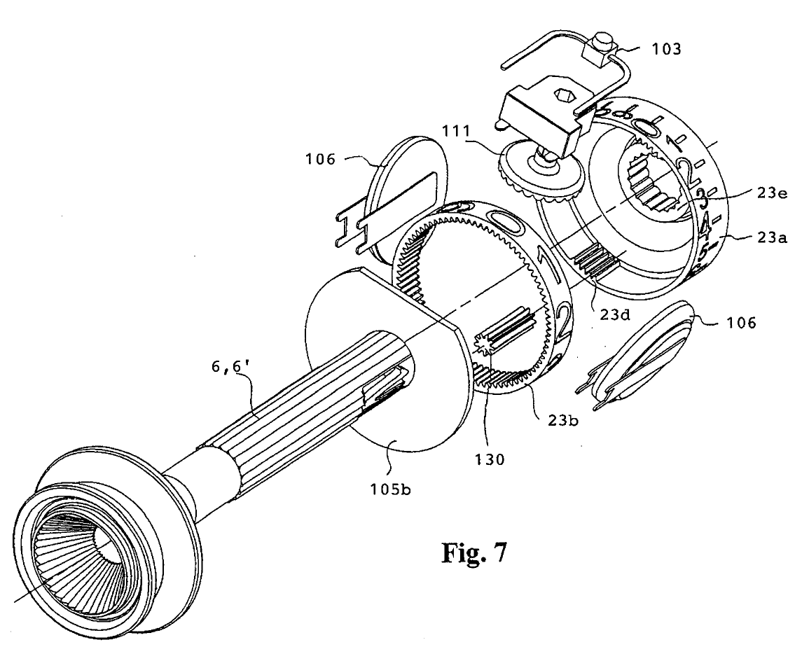

Reference 20″ denotes a dose display, which is coupled via a display coupling element or a units counting ring 23a with the coupling input element 6′ and, as illustrated in FIG. 6 for example, is mechanically coupled by a planetary gear or, as illustrated in FIGS. 7-11, with a tens counting ring 23b, and which, as was the case with the display coupling elements 21 and 22, can be connected to the coupling input element 6′ or coupled with the coupling input element 6′ so that it can not rotate. The display coupling element 23 is not able to move in and opposite the direction of the coupling movement X relative to the housing part 4.

FIG. 1A illustrates the housing part 4 with the parts of the injection device accommodated in it in a non-operating state in which the device can be fitted or the product dose set. To prevent the coupling input element 6′ from effecting the rotary driving movement and hold the driving element 25 in the tensed state, an anti-rotation lock is provided between the coupling housing 6′ and the housing part 4. In the retained position of the coupling members 6′, 7′ and 9, the locking action preventing rotation is between a first locking element 24 and a second locking element 34. The locking element 24 is connected to the coupling input element 6′ so that it is prevented from rotating and sliding. The locking element 34 is connected to the housing part 4 so that it is prevented from rotating but is able to move relative to the housing part 4 and to the coupling input element 6′ in and opposite the direction of the coupling movement X. The locking elements 24 and 34 form a ratchet with their end faces in contact with one another in a locking engagement, which permits a rotating movement of the coupling input element 6′ biasing the driving element 25 and prevents a rotating movement in the opposite direction.

The locking element 24 is provided with a thread on an external face for a second function in connection with the dose setting and dispensing operation, the thread axis of which is coaxial with the thread axis of the plunger rod 15. A stop element 27 engages in this thread. The stop element 27 can be moved and is linearly guided parallel with the thread axis of the locking element 24, and is so in an axial groove on the internal casing surface of the housing part 4 in the embodiment illustrated as an example. The locking element 24 acts as an anti-rotation stop for the stop element 27, which restricts the rotary movement of the coupling input element 6′ causing the plunger rod 15 to be driven forwards. It acts as another anti-rotation stop for the stop element 27, which F determines the maximum dose which can be dispensed and set.

To trigger the driving element 25, a trigger element 28 is provided. The trigger element 28 can be moved in translation relative to the housing part 4 in the direction of the coupling movement X, which in the embodiment illustrated is in the forward drive direction V or distal direction, and in rotation about the rotation axis of the coupling input element 6′, which coincides with the thread axis of the plunger rod 15, and is guided by the housing part 4 during both of these movements. Due to the translating movement in the distal direction, the coupling engagement is established between the coupling input element 6′ and the coupling interconnecting element 7′ and the anti-rotation lock between the locking elements 24 and 34 is released, thereby triggering the driving element 25, i.e. the dispensing operation.

In another function, the trigger element 28 acts as a dose metering element. Due to the rotary movement of the trigger element 28 relative to the housing part 4, the product dose which can be dispensed during the next dispensing operation is set by several intermediate elements. From the zero dose position illustrated in FIG. 1A and due to the abutment of the stop element 27 on the anti-rotation stop of the locking element 24 restricting the driving movement of the coupling input element 6′, the dose can be set by turning the trigger element 28 in the direction of the arrow indicating the direction of rotation. The rotating dose setting movement of the trigger element 28 is transmitted to the coupling input element 6′ via an inner part 29, which is connected to or integrally formed with the trigger element 28 so that it can not turn and slide, and a connecting part 33. For transmission purposes, the inner part 29 and the connecting part 33 are engaged with one another to prevent them from rotating and the connecting part 33 is connected to the coupling input element 6′ so that it can not rotate. The connecting part 33 is also connected to the coupling input element 6′ so that it can not move axially. To produce the rotation locking effect, the inner part 29 and the connecting part 33 are provided with internal teeth 29a and external teeth 33a, which engage with one another when the device is in the non-operating state and can be moved axially towards one another.

In a user-friendly arrangement, the trigger element 28 is disposed in the proximal end region of the housing part 4. Its outer sleeve part surrounds the housing part 4. A base of the trigger element 28 forms a distal end of the injection device. To set the dose, the trigger element 28 is operated in the manner of a rotating knob and for this purpose has knurling on its external casing surface. For the triggering function, it is operated in the manner of a push-button.

Projecting out from the inner part 29 towards a proximal end face of the connecting part 33 is a stop element 29b. When the device is in the non-operating state, a slight distance is left free between the connecting part 33 and the stop element 29b, which is sufficiently big enough so that the triggering movement of the trigger element 28 causes the anti-rotation lock between the inner part 29 and the connecting part 33 to be released before the stop element 29b terminates the relative movement of the trigger element 28 relative to the connecting part 33 due to contact with the stop.

The second locking element 34 is biased into the locking engagement with the locking element 24 by a locking spring 31. To this end, the locking spring 31 is supported on the on the locking element 34 in the direction of the coupling movement X and on a housing part 30 fixedly connected to the housing part 4 in the direction opposite the coupling movement X. Another spring 32 disposed between the inner part 29 and the locking element 34 biases the trigger element 28 relative to the locking element 34 into a proximal end position.

In the non-operating or rest state illustrated in FIG. 1A, the user sets the dose by turning the trigger element 28 in the direction of rotation indicated by the arrow. During this rotating dose setting movement, the trigger element 28 drives the connecting part 33 with it via the anti-rotation lock 29a, 33a, which in turn drives the coupling input element 6′, which therefore effects the same rotating dose setting movement as the trigger element 28. Due to the rotation of the coupling input element 6′, the driving element 25 is biased. The stop element 27 migrates in the engagement with the thread of the locking element 24 from the stop of the thread determining the zero dose in the direction of the stop determining the maximum dose.

If the user has inadvertently set too high a dose, he can correct the dose by turning back the coupling input element 6′. To correct the dose, he pulls the trigger element 28 in the proximal direction. When the device is in the non-operating state, the inner part 29 and the locking element 34 are in a driving engagement with respect to a movement in the proximal direction. The corresponding drivers are denoted by references 29c and 34a. The driver 29c formed by the inner part 29 and the driver 34a formed by the locking element 34 engage behind one another and form a hooked arrangement to move the trigger element 28 in the proximal direction. By pulling on the trigger element 28, therefore, the locking element 34 is likewise moved in the proximal direction and is thus released from the locking engagement with the locking element 24. As soon as the anti-rotation lock is released, the user can correct the dose by the translating dose setting movement of the trigger element 28, and the engagement of the inner part 29 with the connecting part 33, which is maintained as before, prevents any rotation. As soon as the user releases the trigger element 28, it snaps back in the distal direction with the locking element 34 under the action of the locking spring 31 and the locking element 34 is returned to the locking engagement with the locking element 24 as a result. During the return movement, the user expediently continues to hold the trigger element 28 firmly, but only to prevent relative movements in the housing part 4. In principle, however, he can simply let it snap back.

After setting the desired dose, the device is placed at the desired point of the skin for administering purposes and the injection needle inserted. To insert the needle, the user must push the trigger element 28 in the distal direction. A protective needle guard, not illustrated in the drawings, is appropriately coupled with the trigger element 28. As soon as the injection needle is positioned, the driving element 25 can be triggered by applying more pressure to the trigger element 28 and this causes the dispensing process. During the second phase of the triggering movement of the trigger element 28, which is needed following insertion, the trigger element 28 and hence the inner part 29 are pushed against the pressure of the spring 32 relative to the connecting part 33 further in the distal direction so that the anti-rotation lock 29a, 33a is released. The trigger element 28 is able to rotate idly. As soon as the anti-rotation lock 29a, 33a is released, the stop element 29b moves into an abutting contact with the connecting part 33. During the third phase of the triggering movement which now ensues, the trigger element 28 presses the connecting part 33 via the stop element 29b and hence the coupling input element 6′ in the direction of the coupling movement X, which in the embodiment illustrated is the forward drive direction V. Under the effect of the spring force of the locking spring 31, the locking element 34 follows this movement until it makes an abutting contact with the housing part 4. Before the locking element 34 reaches the abutting position, the coupling input element 6′ establishes the coupling engagement with the coupling interconnecting element 7′. The coupling input element 6′ pushes the coupling interconnecting element 7′ out of the frictional locking engagement with the uncoupling element 11′. Once the locking engagement between the conical surfaces of the two elements 7′ and 11′ has been released and hence the coupling engagement fully established, the locking element 34 moves into abutment with the housing part 4. During the final phase of the triggering movement which now ensues, the trigger element 28 pushes the locking element 24 out of the locking engagement with the locking element 34.

As soon as the anti-rotation lock formed by the locking elements 24 and 34 has been released, the rotary driving movement of the coupling input element 6′ is initiated due to the driving force of the driving element 25 and is transmitted to the coupling output element 9 via the coupling engagement. Due to the fact that it is guided in the linear guide 4a and prevented from rotating, the plunger rod 15 moves in the forward drive direction V in the threaded engagement with the coupling output element 9 and product is dispensed. This dispensing movement is terminated due to the abutment of the stop element 27 on the stop of the thread of the locking element 24 defining the zero dose.

FIG. 1B illustrates the injection device in the situation of setting a zero dose in the coupled state after the anti-rotation lock has been released, i.e. after the trigger element 28 has completely effected the triggering movement. The trigger sequence described above advantageously takes place automatically whilst pressure is continuously being applied to the trigger element, from the moment of insertion to the complete dispensing of the set dose; for insertion purposes, the housing must be pressed against the skin surface.

FIG. 1C illustrates the injection device after the reservoir 2 has been emptied. The housing part 1 has already been removed from the housing part 4. The plunger rod 15 has assumed its farthest distal position. The uncoupling element 11′ is blocking the coupling input element 6′ in the position pulled back from the coupling interconnecting element 7′. The way in which the uncoupling element 11′ operates corresponds to that of the other examples of embodiments. Unlike the first two embodiments described as examples, the housing part 1 and the uncoupling element 11′ do not engage directly with one another to form a guide, but do so via an adapter structure 40. The adapter structure 40 is a sleeve which is secured in the housing part 4 so that it is fixed in the connecting portion in and opposite the direction of the coupling movement X but can rotate about the central longitudinal axis of the housing part 4. The adapter structure 40 forms a guide curve 40a, in the form of a either cut-out or orifice in its casing surface facing the uncoupling element 11′. The guide curve 40a has the contour of a threaded portion. The length as measured around the circumference and the pitch of the guide curve 40a measured by reference to the central longitudinal axis of the housing part 4 are dimensioned so that for a quarter to half turn of the adapter structure 40 relative to the uncoupling element 11′, it is moved into the uncoupled position illustrated in FIG. 21. The movement of the uncoupling element 11′ has an axial length corresponding to the length X of the full coupling movement. To generate the axial movement, the uncoupling element 11′ locates in the guide curve 40a by means of its locating element 12. In this respect, reference may be made to the explanations given above.

When the housing parts 1 and 4 are connected, the adapter structure 40 forms a linear guide for the housing part 1. The housing part 1 is pushed into the adapter structure 40, in which case a slight friction is generated and accordingly a slide guide for the housing part 1. The housing part 1 can not be rotated relative to the adapter structure 40 about the central longitudinal axis of the housing part 4. The corresponding anti-rotation engagement is established immediately at the start of the pushing action of the housing part 1 into the adapter structure 40. Once the housing part 1 has been pushed so that it abuts with the housing part 4, namely its coupling mount at 4a, the housing part 1 is turned relative to the housing part 4 and during this rotating movement drives the adapter structure 40 with it until the locating element 12 of the uncoupling element 11′ moves into abutment with the end of the guide curve 40a. The rotating movement of the housing part 1 is not possible until it has reached its axial abutting position, for which purpose an active anti-rotation lock may also be provided between the housing parts 1 and 4 until the abutting position is reached.

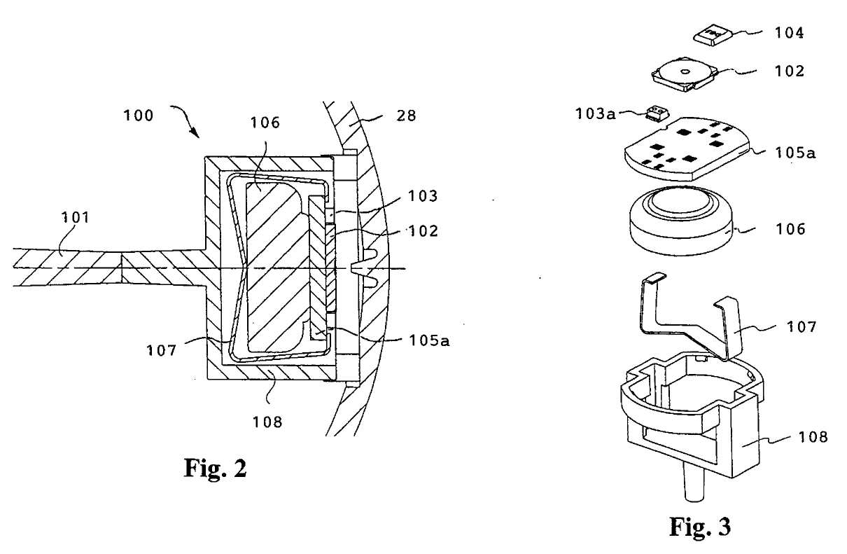

FIG. 2 and FIG. 3 show a cross-section of a life timer 100 integrated in the trigger element or dose setting knob 28, which is connected to the threaded rod or plunger rod 15 by a connecting piece 101. If the ampoule inserted in the injection device is full, the switch 102 of the life timer can be operated by the threaded rod 15 and connecting piece 101 so that the LED 103a is briefly lit in green. To evaluate the signals generated by the contact switch 102 and activate the LED 103a, a chip 104 is provided on the circuit board 105a. The requisite power is supplied by a battery 106, which is secured to the circuit board 105a by means of a contact blade 107, and the life timer 100, in some embodiments comprising components 102-107, is mounted in the compartment 108. The elements of the first embodiment of the life timer illustrated in cross-section in FIG. 2 are illustrated individually in FIG. 3. Integrated in the chip 104 is a time detection unit, which is started by a signal from the switch 102 the first time a deployment operation is detected. On expiry of the maximum service life of 3 years, for example, stored in the chip, the LED 103a is activated so that it emits red light or flashes, to indicate to a user that he should no longer use the injection device and must replace it. In addition or as an alternative, the number of ampoule changes may also be used as a means of measuring or determining the end of the service life, in which case these will also be detected by the switch 102.

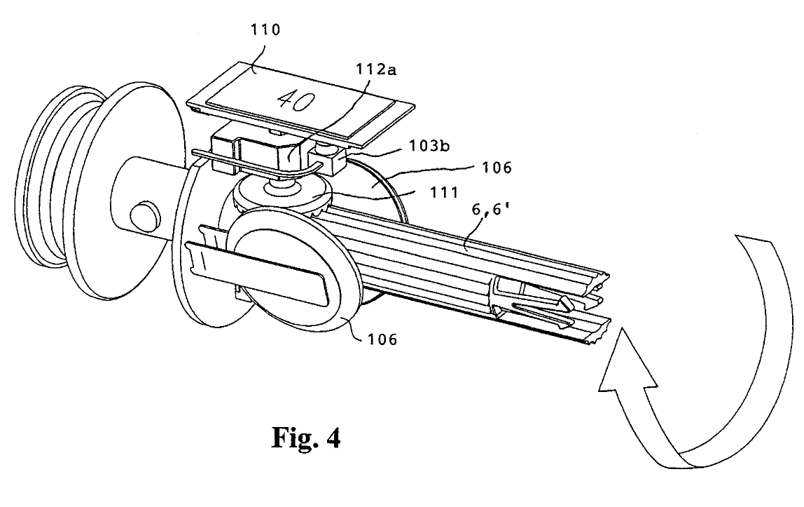

FIG. 4 illustrates an electronic unit integrated in the injection device with an LCD display 110, which is illuminated with green light from the rear face by an LED 103b when the injection pen is still within its service life and with a red light when the injection pen is operated outside of a service life which is fixed or determined on the basis of selected conditions. A spur gear or ring gear 111 meshes with a set of teeth or the coupling input element and transmits a rotating movement of the coupling sleeve to the encoder 112a, and the signal generated by the encoder 112a is processed by an electronic system and output for the display on the LCD 110 to be read by the user. Since the coupling sleeve or the coupling input element is also moved during the setting operation and during a dispensing operation, it is always possible for the encoder 112 coupled with the coupling sleeve via the spur gear 111 to detect which dose still has to be made available for dispensing from the injection device, so that the display on the LCD 110 runs backwards during the dispensing operation.

Apart from the coupling sleeve, a sensor or encoder may also be coupled or connected directly or indirectly to other elements involved in the dose setting or dispensing operation, such as a counting ring 23a, 23b, the locking element 24 or the connecting part 33.

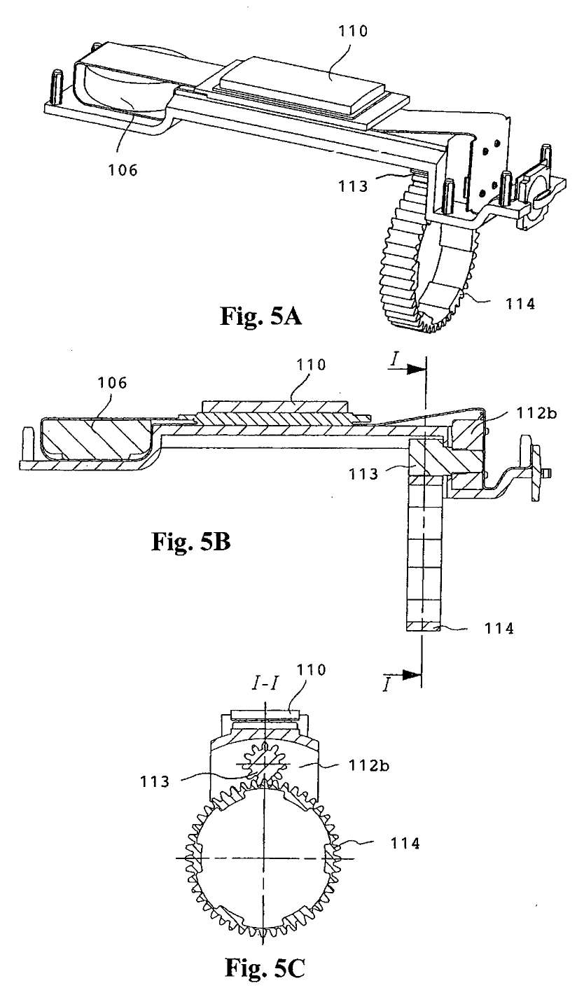

FIG. 5A is a perspective view illustrating another embodiment of an electronic real-time display illustrated in cross-section in FIG. 5B and in a plan view in FIG. 5C, and a ring gear 114 is disposed on a setting or dose metering element of the injection device so that it can not rotate, although this is not illustrated in the drawing, in which a gear 113 coupled with a stop-free encoder or rotary coder 112b engages to detect a rotating movement of the dose setting element and display a dose corresponding to the rotating movement on the LCD display 110. The encoder 112b could also be coupled with the coupling input element by a ring gear, as illustrated in FIG. 4.

FIG. 6 illustrates an embodiment of a mechanical, coaxially disposed real-time display with a units counting ring 23a and a tens counting ring 23b of the type described above. The drive disc 120 connected to the coupling sleeve 6, 6′ (not illustrated in FIG. 6) has three bearings 120a for planet gears 121, which are driven by the drive disc 120. The planets 121 drive the units counting ring 23a, which has external teeth 23c serving as a sun gear, which is coaxially offset and has a smaller diameter than the units counting ring 23a. Provided on a ring gear 122 is a bearing for a coupling gear 123 which engages in the inner teeth 23d on only a part region of the units counting ring 23a after every full revolution of the units counting ring 23a to move the tens counting ring 23b round by a further 36 degrees. The ring gear 123 serves as a support for the unit illustrated in FIG. 6 and is retained in the housing of the injection device. The direction of rotation of the units counting ring 23a and the tens counting ring 23b corresponds to the direction of rotation of the drive disc 120 coupled or connected to the coupling sleeve, thereby enabling a mechanical real-time display to be implemented, which continuously counts any corrections made to the setting during the setting operation and carries on counting during the dispensing operation and displays the quantity of the dose still available for dispensing after setting in real time.

FIG. 7 illustrates an embodiment in which the concept described in connection with FIG. 4 is provided not with an electronic but with a mechanical display. This being the case, the coupling sleeve is inserted through a circuit board 105b and has teeth on the external surface, in which the spur gear 111 engages, as described in connection with FIG. 4. The external teeth of the coupling sleeve are coupled with a gear acting as a driver 130, which transmits the rotating movement of the coupling sleeve to internal teeth 23e of the units counting ring 23a, and the units counting ring 23a is coupled with the tens counting ring 23b by means of the internal teeth 23d provided on only a part of the internal circumference, as described in connection with FIG. 6 to implement a real-time display. The units and tens counting rings 23a and 23b are transparent so that a red or green light emitted by the LED 103 to the display will indicate a to user reading the setting on the counting rings 23a, 23b whether the injection device can still be used (green) or whether the service life has expired (red).

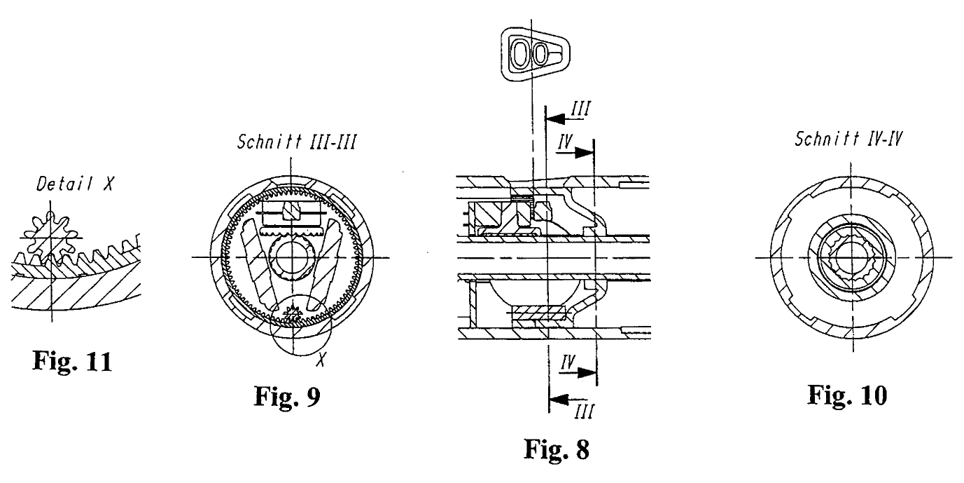

FIG. 8 illustrates the device shown in FIG. 7 in cross-section in the assembled state, a section being indicated by line III-III indicated in FIG. 9 and line IV-IV indicated in FIG. 10. The detail marked X in FIG. 9 is illustrated in FIG. 11.

Embodiments of the present invention, including preferred embodiments, have been presented for the purpose of illustration and description. They are not intended to be exhaustive or to limit the invention to the precise forms and steps disclosed. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. An injection device comprising a regulating element, a sensor, an electronic evaluation system connected to the sensor, and a display connected to the evaluation system for displaying information, wherein the sensor is arranged in the injection device or coupled to operational elements of the device whereby, during a delivery or operational process, the dose still to be delivered can be assessed.

2. A method for displaying a dose of a substance to be delivered from an injection device, the method comprising the steps of detecting and displaying the dose during a regulating or operating process associated with the injection device, wherein a dose of the substance still to be delivered is displayed during a delivery process.

3. An injection device, containing a substance to be dispensed or in which an ampoule can be inserted, comprising operational elements, a setting element for setting a dose or quantity of the substance to be dispensed, a sensor for detecting a set dose, an electronic evaluation unit connected to the sensor and a display connected to the electronic evaluation unit and able to display the set dose, wherein the sensor is associated with the injection device such that a dose still to be dispensed can be sensed during a dispensing operation.

4. The injection device as claimed in claim 3, wherein the sensor is one of a rotary sensor or encoder, which can be coupled with at least one of the operational elements of the injection device, the movement or rotation of which is used in determining the dose set or still to be dispensed.

5. The injection device as claimed in claim 4, wherein the at least one operational element is one of a coupling sleeve or coupling element.

6. The injection device as claimed in claim 5, further comprising an electronic display.

7. The injection device as claimed in claim 4, further comprising a mechanical display comprising one, two or more coaxially disposed counting rings.

8. The injection device as claimed in claim 7, wherein at least two counting rings are coupled by a planetary gear.

9. The injection device as claimed in claim 3, further comprising at least one of an electronic and mechanical display illuminated by a light source.

10. The injection device as claimed in claim 9, wherein the light source is one of an LED or OLED.

11. A method of displaying a dose of a substance to be administered, set on an injection device or still to be dispensed from the injection device, the method comprising the steps of detecting and displaying the dose during a setting operation, wherein the quantity or dose of the substance to be administered which still has to be dispensed is also displayed during a dispensing operation.

12. The method as claimed in claim 11, further comprising the step of one of electronically or mechanically re-setting when an end of the dispensing operation or a change of ampoule is detected.

13. The method as claimed in claim 11, further comprising the step of switching on at least one of an electronic display and a light source of at least one of an electronic and mechanical display when it is detected that the injection device is being operated.

14. The method as claimed in claim 13, wherein the at least one of the electronic display and light source is switched off when a predefined period has elapsed after a deployment of the injection device has been detected or when it is ascertained that the injection device has not been deployed within a predefined period of time.
